(19) United States
(12) Patent Application Publication
(10) Pub. No.: US 2016/0324818 A1
(43) Pub. Date: Nov. 10, 2016

(54) DIET FOOD COMPOSITION INCLUDING DHA TO REDUCE SHORT-TERM AND LONG-TERM LOW-GRADE INFLAMMATION

(71) Applicant: PRI S.A., Clemency (LU)

(72) Inventor: ABDELHADI ZAHOUANI, ARGENTEUIL (FR)

(73) Assignee: PRI S.A., Clemency (LU)

(21) Appl. No.: 15/109,249

(22) PCT Filed: Dec. 29, 2014
(86) PCT No.: PCT/EP2014/079391

§ 371 Date: Jun. 30, 2016

(30) Foreign Application Priority Data
Dec. 30, 2013 (FR) 1363718

(51) Int. Cl. A61K 31/202; A61K 31/70; A23L 33/12; A23L 33/17; A23L 33/125; A61K 38/00; A61K 9/48

(52) U.S. Cl. CPC A61K 31/202; A61K 38/00; A61K 31/70; A61K 9/48; A23L 33/17; A23L 33/125; A23L 33/12; A23V 2002/00

(57) ABSTRACT

Ready-to-use diet food composition to reduce low-grade inflammation and stabilize weight loss in the long term in an obese or overweight patient comprising, as a proportion of the total weight of the composition:
50% to 80% by weight of protein;
2% to 20% by weight of carbohydrates; and,
2% to 15% by weight of lipids;

said lipids including 0.25% to 2% by weight of docosahexaenoic acid (DHA) relative to said weight of lipids.

The invention also concerns ready-to-use portions of diet food, a kit comprising a food portion and DHA capsules as well as the use of DHA to prepare a food composition or food portions according to the invention.

### FIELD OF THE INVENTION

The field of the invention is that of diet food compositions. More specifically, the invention relates to a diet food composition to be consumed in a very-low-calorie type diet to reduce low-grade inflammation over the short term (between one and three months) and long term (more than one year).

### PRIOR ART

According to the WHO figures, obesity has doubled since 1980 and overweight concerns more than 1.4 billion individuals aged 20 and above (estimates for 2008) (http://www.who.int/mediacentre/factsheets/fs311/fr/index.html). According to estimates for 2011, approximately 40 million children under five are overweight. Now, according to WHO figures, approximately 3 million deaths are directly related to overweight and obesity, which makes them the fifth highest risk factor of death in the world. At present, it would seem that these figures are on the rise.

The notions of obesity and overweight are commonly assessed by the body mass index (BMI) which is the ratio between the weight in kilograms and the square of the height in meters: BMI=mass (kg)/height² (m). A patient is considered to be overweight when his BMI is greater than or equal to 25. Obesity is confirmed when the BMI is greater than or equal to 30 and morbid obesity is determined by a BMI greater than or equal to 35 or 40.

Obesity and overweight have a variety of causes: genetic mutations, hormone imbalance, etc. However, they are most usually linked to bad habits, namely a calorie intake appreciably greater than the subject's energy expenditure.

Obesity and overweight also cause various problems: joint problems, breathlessness, difficulty in moving, excessive fatigue, depression due to poor self-image, feelings of being rejected by society, etc. In addition to these problems, obesity and overweight are also considerable risk factors for the patient's health and especially for cardiovascular illnesses, hypertension, certain forms of cancer and type 2 diabetes. In addition, the expenditure on care for these patients and their pathologies amounts to about 2%-7% of expenditure by health systems in the developed countries. As a consequence, obesity and overweight have become problems of public health the world over.

At the molecular level, it has recently been discovered that obesity is linked to a moderate but chronic increase in the flow rates of inflammation mediators such as interleukin 6 (IL6), fibrinogen, the reactive protein C (PRC), the “tumor necrosis factor alpha” (TNFα), serum amyloid A (SAA), etc. This phenomenon is symptomatic of chronic and moderate inflammation or low-grade inflammation.

Numerous studies have correlated an increase in the TNFα rate and IL6 with an increase in body fat mass, henceforth allowing the scientific community to consider obesity to be a chronic inflammatory condition like other pathologies frequently associated with obesity: type 2 diabetes and atherosclerosis for example.

It can also be seen that certain slimming diets proposed slightly reduce the concentration in PRC, TNFα and IL6 in an obese patient. However, these observations must be taken with precaution: the effects of a very-low-calorie diet have been studied only for short periods among obese patients having type 2 diabetes. In addition, results of studies have shown that, despite considerable weight loss and a major reduction of the waistline indicating a loss of body fat mass, the levels of IL6 and TNFα and other pro-inflammatory cytokines do not return to a normal level. Chronic inflammation therefore persists despite weight loss and loss of body fat mass. Now, weight increase and chronic inflammation are closely linked. This persistence of low-grade inflammation is the cause of the rebound or weight resumption seen in obese patients or overweight patients at the end of a very-low-calorie diet.

Now it is important to limit this regaining of weight, often called “the yo-yo effect” or the “rebound effect”. The rebound effect is the cause of obesity and episodes of depression aggravated by feelings of frustration and social isolation caused by failures of diets. The rebound effect is in major part related to the persistence of the state of chronic inflammation which is not resolved by diet alone. In addition, the rebound effect discourages the patient who feels a sort of frustration and a sense of failure after regaining weight despite great effort. In addition, the patient then tries to again lose the weight that he has put back by following a diet again. Now, the organism ultimately resists the performance of consecutive diets. In addition, certain patients force themselves to adopt long-term low-calorie diets and therefore inflict much food privation on themselves in their anxiety about resuming their food habits. These situations aggravate the social isolation and the fragile psychological state of obese patients.

### GOALS OF THE INVENTION

The invention is aimed especially at overcoming these drawbacks of the prior art.

More specifically it is a goal of the invention, in at least one embodiment to provide a diet food composition to reduce low-grade inflammation among overweight or obese patients.

It is another goal of the invention, in at least one embodiment, to provide individual food portions to be consumed by the subject in the context of very-low-calorie type diet.

It is also a goal of the invention, in at least one embodiment, to provide a food composition or individual food portions to limit the risk of regaining weight.

### SUMMARY OF THE INVENTION

These goals as well as others that shall appear below are achieved by means of a ready-to-use diet food composition to reduce low-grade inflammation among obese or overweight patients comprising, as a proportion of the total weight of the composition:
50% to 80% by weight of protein;
2% to 20% by weight of carbohydrates; and,
2% to 15% by weight of lipids;

said lipids including 0.25% to 2% by weight of docosahexaenoic acid (DHA) relative to said weight of lipids.

Thus, the invention relies on a wholly novel and original approach, proposing a food composition comprising a high percentage of a particular polyunsaturated fatty acid of the family known as the omega 3 family: docosahexaenoic acid (DHA). It is known that omega 6 has a pro-inflammatory role while omega 3 tends to diminish the level of inflammation markers in the body. The specialists generally recommend keeping the omega 6/omega 3 ratio below 5. Now the ratio observed in the food taken by overweight or obese subjects is generally greater than 10. The quantities of omega 3 recommended to restore the ratio between omega 6/omega 3 to a value of below 5, combat weight regain and protect the cardiovascular system are impossible to obtain solely through food intake. The inventors have therefore designed a diet food composition to cover this intake of omega 3. Thus, the food composition according to the invention diminishes the concentration of inflammation markers in the short term and the long term, i.e. starting from the first month of consumption and over several years, and diminishes or even eliminates low-grade inflammation among obese or overweight patients. The resolution of the inflammatory state facilitates weight loss while at the same time preventing the phenomenon of rebound at the end of the diet. The slimming diet is therefore more efficacious.

The originality of the present invention therefore lies in the fact that the compositions and the food portions not only enable weight loss but also prevent any regaining of weight by rebound effect through the resolution of low-grade inflammation. It is important to distinguish between the different “types” of inflammation. The term “low-grade chronic inflammation” is understood to mean inflammation spread throughout the subject's organism without any precise inflammatory site and without swelling, heat, redness and pain. No inflammatory site is apparent. The cells of the immune system and the inflammatory substances are present in a diffuse manner in the organism and damage the subject's healthy cells. These inflammatory substances can be measured in the blood and the cells of the immune system observed under a microscope. This inflammation can be caused by a persistent initial infection following a viral infection for example. It can also be caused by an excess of sugar and/or fat in the diet. Obesity has recently started being considered to be a low-grade systemic inflammation, just like many pathologies associated with it such as atherosclerosis, type 2 diabetes etc.

Hitherto, no publication has mentioned food compositions or food portions that can be used to prevent the rebound effect or the yoyo effect by reducing low-grade systemic inflammation.

In one advantageous embodiment, the composition according to the invention comprises the following, relative to the total weight of the invention:
70% to 80% by weight of protein
4% to 6% by weight of carbohydrates; and
2% to 5% by weight of lipids; said lipids including 0.25% to 2% by weight of docosahexaenoic acid (DHA) relative to said weight of lipids.

Advantageously, the composition according to the invention is used by the patient for a period of 30 to 180 days, preferably 60 to 180 days, and more preferably 90 to 180 days. This duration can be determined by the physician according to the results of tests on several of the patient's parameters such as weight loss, waistline measurements and levels of lipids and inflammation markers. However, it is preferable to consume these portions for at least 30 days. Indeed, although a loss of weight can be observed at the end of the 2 to 3 weeks and even from the very first days of the diet, it is preferable to consume the portions according to the invention for at least one month in order to observe the first effects of the DHA on the resolution of chronic low-grade inflammation. Scientific studies have shown that the mechanism of the resolution of inflammation related to the secretion of the lipid mediators of inflammation derived from fatty acids requires several days to be activated (C. N. Serhan, Novel Lipid Mediators and Resolution Mechanisms in Acute Inflammation: To Resolve or Not ?, The American Journal of Pathology, vol. 177(4), 4 Oct. 2010, pp: 1576-1591). More specifically, the more prolonged the consumption of the portions according to the invention, the lower the level of secretion of pro-inflammatory markers and the greater the extent to which the anti-inflammatory markers are expressed.

An object of the invention furthermore concerns an individual diet food portion to reduce low-grade inflammation comprising:
12.5 g to 20 g, preferably 14 g to 16 g, of protein and,
0.5 g to 5 g, preferably 1 g to 5 g of carbohydrates;
0.25 to 6 g, preferably 0.5 g to 4 g of lipids; said lipids including up to 80 mg of DHA.

The individual portions can therefore constitute a snack, a meal, or a part of a meal. It is planned, in the context of a diet of the very-low-calorie type under medical supervision, to vary the daily quantity of DHA taken by the patient. The term “very-low-calorie diet” is understood to mean a diet in which the patient's daily calorie intake is limited to a maximum of 800 kcals per day.

In the first two weeks of his diet, the patient starts taking 500 mg-600 mg of DHA per day.

The food portions according to the invention can take the form of a powder to be mixed with water, for example to obtain a broth, a soup, a hot or cold drink such as chocolate, coffee, fruit juice, or an omelet, etc.

The food portions according to the invention can also take the form of ready-to-eat foods such as (not exhaustively) cereal bars or flavored bars, pancakes, biscuits, crepes, sauces, breakfast cereal. It is important for the subject to keep foods in a form identical or close to that of conventional foods so that he or she does not have to give up on their diet. This also makes it possible not to cut oneself off from one's social environment by being unable to share a meal with friends or family. The individual portions therefore also facilitate dosing, i.e. the quantity of food that is the simplest for the patient to calculate since all he or she needs to do is to count the number of portions to be eaten during a meal. Indeed, certain individuals gain weight or do not manage to slim in a balanced manner because they tend to consume ever larger quantities of food without realizing that they are exceeding their daily energy needs. Finally, the individual portions are easy to transport: the subject can thus carry a bar to serve as a snack during the day, so that he can satisfy his hunger without deviating or so that he can prepare lunch at his workplace.

Another advantage of the food portions according to the invention is that they make it easier to calculate the daily dose of protein and of DHA to be consumed by the patient. Indeed, the quantity of DHA that a practitioner or dietician prescribes can vary according to the patient's needs, his resistance to the consumption of these compositions or food portions, the weight loss envisaged. In this case, it is worthwhile for a patient to be able to rapidly calculate his daily dose of DHA quite simply on the basis of the number of portions consumed during the day. He is therefore free to distribute them according to his lifestyle. It is thus simpler for the patient to follow the daily dose of DHA prescribed by the doctor according to the number of portions recommended daily.

It must be noted that this latter advantage is also obtained with a food composition according to the invention. For example, the food composition according to the invention can take the form of powder in bulk accompanied by a measuring spoon. The patient then has to count the number of spoons of composition consumed in the day.

Advantageously, the individual diet food portion according to the invention comprises 1 mg to 80 mg of DHA, preferably 10 mg to 50 mg of DHA, in a more preferred way 25 mg to 50 mg of DHA and in a manner preferred among all, 50 mg of DHA

According to the invention, the individual diet food portion has a weight of 5 mg to 300 mg, alternatively 15 g to 300 g, preferably 15 g to 200 g, and in a more preferred manner 20 g to 125 g.

The portions according to the invention can take the form of batches comprising for example several elements of 5 g or 10 g for example a small packet of biscuits.

Preferably, the individual food portion is made out of culinary ingredients at least one of which contains DHA. It is preferable to formulate individual food portions out of natural food sources rather than to include a synthetic form of DHA. Indeed, the incorporation in the form of culinary ingredients makes it easier to formulate the portions according to the invention. In addition, it is important that the food portions should be flavorsome and pleasant to taste in order to facilitate compliance with the diet.

Advantageously, the portion according to the invention is used for a period of 30 to 180 days by the patient, preferably 60 to 180 days, and more preferably 90 to 180 days. The duration of consumption of the individual food portions depends on the initial weight and the number of kilos to be lost. However, it is preferable to consume the portions for at least 30 days. Indeed, although a loss of weight can be observed at the end of 2 or 3 weeks, it is preferable to consume portions according to the invention for at least one month in order to note the first effects of DHA on the resolution of chronic low-grade inflammation. More specifically, the longer the time in which the portions according to the invention are consumed, is prolonged, the fewer the inflammation markers secreted and the greater the extent to which the anti-inflammatory markers will be expressed.

In one advantageous embodiment, said culinary ingredient containing DHA is chosen from amongst egg yolk, fish oil, seaweed extract, mushrooms, krill or a combination of at least two of these ingredients. These ingredients are major natural sources of the DHA.

In one promising embodiment, said protein is protein chosen from among milk proteins, whey or milk serum, soya, egg or a combination thereof.

According to the invention, the ready-to-use dietary food composition can be obtained by mixing a portion as described above with water. Thus, the dietary food composition is a meal obtained by mixing a food portion such as soup, or a freeze-dried beverage with water. Sometimes, certain mixtures can be heated.

The term “ready to use” refers to a food composition that does not require cooking or cooking time beyond five minutes in the microwave oven.

The ready-to-use dietary food composition according to the invention can also be obtained by associating an individual food portion according to the invention with at least one DHA capsule, said capsule containing 100 mg to 300 mg of DHA. Thus, the patient can also choose to maintain a more traditional diet and take the recommended daily dose of DHA by associating an individual portion according to the invention with DHA capsules to form a food composition according to the invention.

Another object of the invention is a kit for a very-low-calorie type of diet comprising at least one dietary food portion according to the invention and at least one DHA capsule. According to the invention, such a kit is aimed at reducing low-grade inflammation in an obese or overweight patient.

An object of the invention is also the use of DHA to prepare a dietary food composition to reduce low-grade inflammation in an obese or overweight patient.

Advantageously, the use of DHA enables the preparation of a dietary food composition comprising by weight, relative to the total weight of the composition:
50% to 80% by weight of protein;
2% to 20% by weight of carbohydrates; and
2% to 15% by weight of lipids;

said lipids including 0.5% to 15% of docosahexaenoic acid (DHA) by weight relative to said weight of lipids.

Advantageously, the use of DHA according to the invention enables the preparation of a dietary food composition to reduce low-grade inflammation in obese or overweight patients comprising, relative to the total weight of the composition:
70% to 80% by weight of protein;
4% to 6% by weight of carbohydrates; and
2% to 5% by weight of lipids;

said lipids including 0.25% to 15% of docosahexaenoic acid (DHA). In one embodiment, said lipids include 0.25% to 2% of DHA relative to the total weight of the lipids. In another advantageous embodiment, said lipids included 10% to 15% of DHA.

### LIST OF FIGURES

Other characteristics and advantages of the invention shall appear more clearly from the following description of a preferred embodiment, given by way of a simple illustratory and non-exhaustive example, and from the appended figures of which:

FIG. 1 is a graph presenting the progress of weight loss in patients having followed a very-low-calorie diet with or without DHA supplements on D-30, D-60 and D-180;

FIG. 2 is a graph representing the progress of the waistline among patients in two groups studied on D-30, D-60 and D-180;

FIG. 3 is a graph illustrating the progress of the pro-inflammatory marker PRCus between the two groups;

FIG. 4 is a graph illustrating the progress of the leptin level in two groups;

FIG. 5 is a graph illustrating the progress of the PD1 level in the two groups;

FIG. 6 is a graph presenting the variations in the 14-HDoHE rate in the two groups studied;

FIG. 7 is a graph illustrating the variation, for each of the groups of patients, of the concentration of pro-inflammatory molecules and anti-inflammatory molecules between D-0 and D-180;

FIG. 8 is a graph presenting the evolution of the ARA/DHA ratio for each group of patients on D-30, D-60 and D-180; and

FIG. 9 is a graph illustrating the progress of the rate of the anti-inflammatory fatty acids index (AIFAI) for each group of patients on D-30, D-60 and D-180.

### DESCRIPTION OF ONE EMBODIMENT OF THE INVENTION

The general principle of the invention relies on the incorporation of a particular omega 3 fatty acid, DHA, in the dietary food compositions in order to reduce or even eliminate low-grade inflammation among obese or overweight patients. According to the invention, the food composition can take the form of an individual food portion, powder in bulk to be mixed in portions with water or in the form of ready-to-consume food such as a bar, a biscuit, a beverage pack, etc.

Below we describe example of food portions according to the invention.

### Example of an Individual Portion According to the Invention: Moka

The portion according to the invention can take the form of a powder to be mixed with cold water to obtain a Moka coffee. The portion has a total weight of 25 g and comprises:
protein: 15 g
lipids: 0.6 g including 50 mg of DHA
carbohydrates: 3.8 g
fibers: 1.7 g
minerals: 1.9 g.

The beverage can also include 1 g to 2 g of mineral salts for the healthy balance of the subject. These will be mineral salts such as those of calcium, phosphorous, sodium, potassium, magnesium, iron, zinc, copper, iodine, manganese, selenium, chromium, molybdenum and fluorine. The beverage can also be enriched with vitamins and essential amino acids.

### Example of an Individual Portion According to the Invention: Apple Cake

The cake has a weight of about 27 g and comprises:
protein: 15 g
lipids 1.6 g including 50 mg of DHA
carbohydrates: 2.9 g
fibers: 5.1 g
minerals: 1 g

The cake also has mineral salts and all the essential amino acids.

### Example of an Individual Portion According to the Invention: Breakfast Cocoa

The portion according to the invention can take the form either of a powder to be mixed with cold water to obtain a cocoa drink or that of an individual beverage ready to be consumed and taken away. The portion has a total weight of 25 g and comprises:
protein: 15 g
lipids: 2.2 g including 50 mg of DHA
carbohydrates: 1.7 g
fibers: 4.2 g
minerals: 1.7 g.

The cocoa beverage according to the invention also has mineral salts including calcium (214.4 mg), phosphorous (171.6 mg), sodium (249.4 mg), potassium (179.2 mg), magnesium (32.5 mg) and iron (1.1 mg).

### Example of an Individual Portion According to the Invention: Chicken Nuggets

Each nugget has a weight of about 27 g and comprises:
protein: 15 g
lipids: 1.9 g including 50 mg of DHA
carbohydrates: 2.5 g
fibers: 4.9 g
minerals: 1 g.

Each nugget has about 545 mg of mineral salts, including calcium, phosphorous, sodium, potassium, magnesium, iron and zinc.

### Example of an Individual Portion According to the Invention: Cheese-Flavored Omelet

The omelet according to the invention takes the form of a 37 g packet of powder which has to be mixed with water before cooking. The powder comprises:
protein: 15 g
lipids: 2.9 g including 50 mg of DHA
carbohydrates: 3.4 g
fibers: 2.1 g
minerals: 1.9 g.

The powder for the omelet also comprises about 830 mg of mineral salts (calcium, phosphorous, sodium, potassium, magnesium, iron and zinc).

### Comparative Results Between Subjects Having Followed a Slimming Diet Including and Not Including DHA

A study under medical supervision was conducted among obese patients (average BMI=33). The subjects were separated into two groups:
a “control” group consuming food compositions similar to those of the invention but without DHA; and
a “study” group consuming food compositions according to the invention comprising DHA.

At the start of the program as well as after 30 days if necessary, then 60 days and at the end of 6 months the subjects underwent a blood test in order to evaluate their levels of pro-inflammatory and anti-inflammatory markers depending on their increase or loss of weight. The markers or molecules indicating the presence of inflammation that were tested for were leucocytes, neutrophils, the reactive protein C (PRC), TNF-α, interleukin 6, leptin, resistin and adiponectin.

The lipid markers tested for, indicating a regression of inflammation or having anti-inflammatory activity are 6kPGF1-alpha (6-keto-prostaglandin F1-alpha), TxB2 (tromboxane B2), PGE2 (prostaglandin E2), LxB4 (lipoxin B4), LxA4 (lipoxin A4), RvD1 (resolvin D1), LtB5 (leukotriene B5), 7MaResin (Maresin 7), Pdx (Protectin dx), LtB4 (leukotriene B4), 18-HEPE (18-hydroxyeicosapentaenoic acid), 15-HETE (15-hydroxyeicosatetraenoic acid), 17-HDoHE (17-docosahexaenoic acid), 14-HDoHE (14-docosahexaenoic acid), 8-HETE (8-hydroxyeicosatetraenoic acid), 12-HETE (12-hydroxyeicosatetraenoic acid) et 5-HETE (5-hydroxyeicosatetraenoic acid). These are lipid mediators derived from DHA.

For the first 60 days of the study, the patients in the study group each received 500 mg of DHA per day solely through the consumption of food compositions and portions according to the invention. Their calorie intake was below 800 kcals per day and was presented chiefly in the form of food compositions or portions according to the invention. To be more precise, the patients consumed five food portions according to the invention during the day, two of them during the main meals where the portions were accompanied by vegetables in order to ensure their DHA intake especially. After 60 days, their calorie intake was gradually increased by the introduction of different groups of food until it reached a calorie level of 1300-2000 kcals per day. The goal was to re-educate the patient in terms of food intake. Traditional foods were gradually re-introduced into the patient's daily food intake in order to avoid deficiency and fatigue as well as to re-educate him or her on the food intake level.

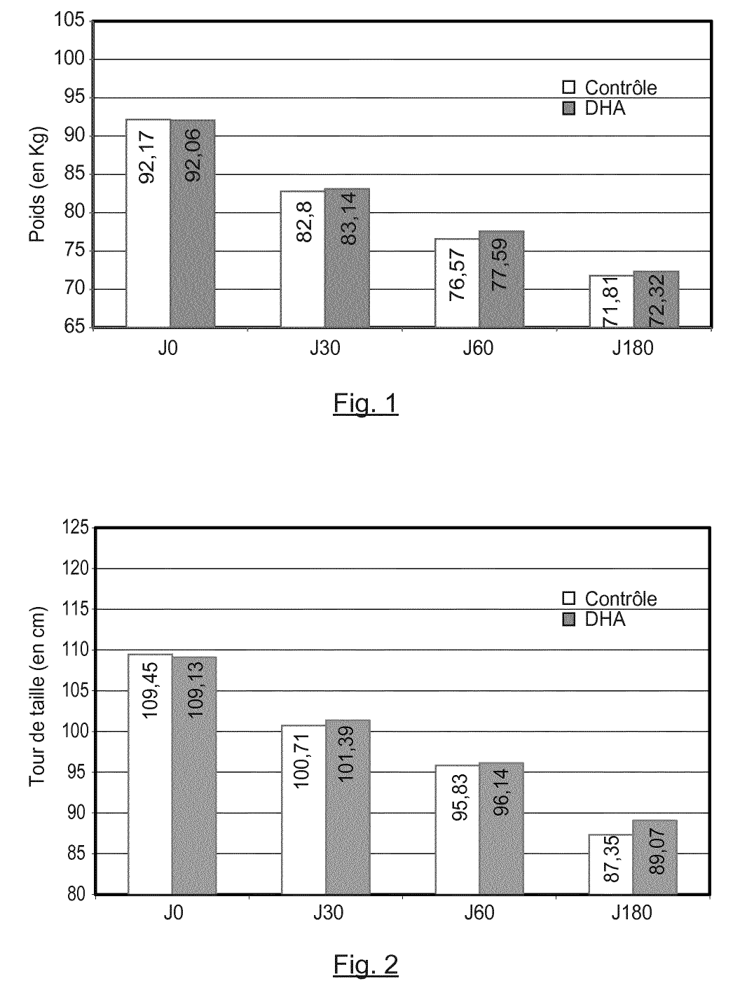

As can be seen in FIG. 1, the subject of the two groups lost 15 kg to 16 kg on an average in a span of 60 days by following the slimming program, and about 20 kg in 6 months (D-180) from the start of the diet on D-0. The progress of the waistline measurements in the two groups of patients is shown in the graph of FIG. 2. The waistline measurement makes it possible to evaluate loss of abdominal fat, a parameter associated with the metabolic syndrome. As can be seen, the subjects of each group lost an average about 15 cm in their waistline measurement at the end of 60 days and between 20 cm to 20 cm after 6 months of a slimming diet. The similarity of the results as regards weight loss and progress of the waistline can be explained by the fact that the patients followed the same low-calorie diet, independently of DHA intake.

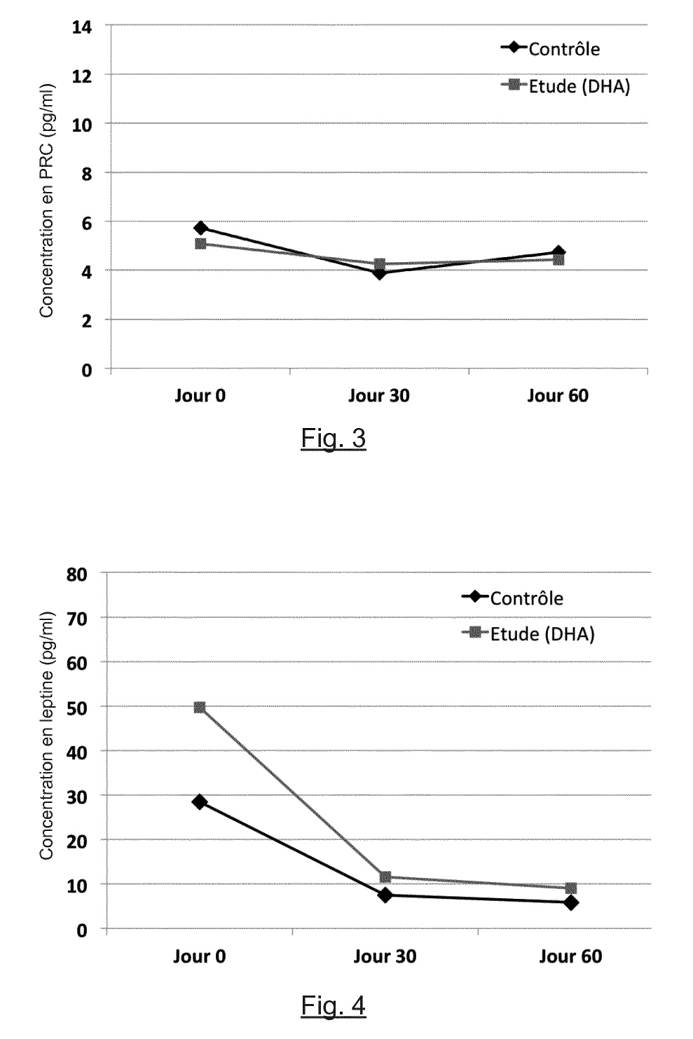

It can be seen from FIGS. 3 and 4 that there was a diminishing of the pro-inflammatory markers. Two inflammation markers associated with obesity were tested for and their levels were measured: these were ultrasensitive reactive protein C (PRCus) and leptin.

The weight loss reduced the overall PRC rate among the subjects of both groups (FIG. 3). More precisely, a reduction of 12% was measured for patients in the control group while a reduction of approximately 18% was obtained for patients having received DHA every day during their slimming diet.

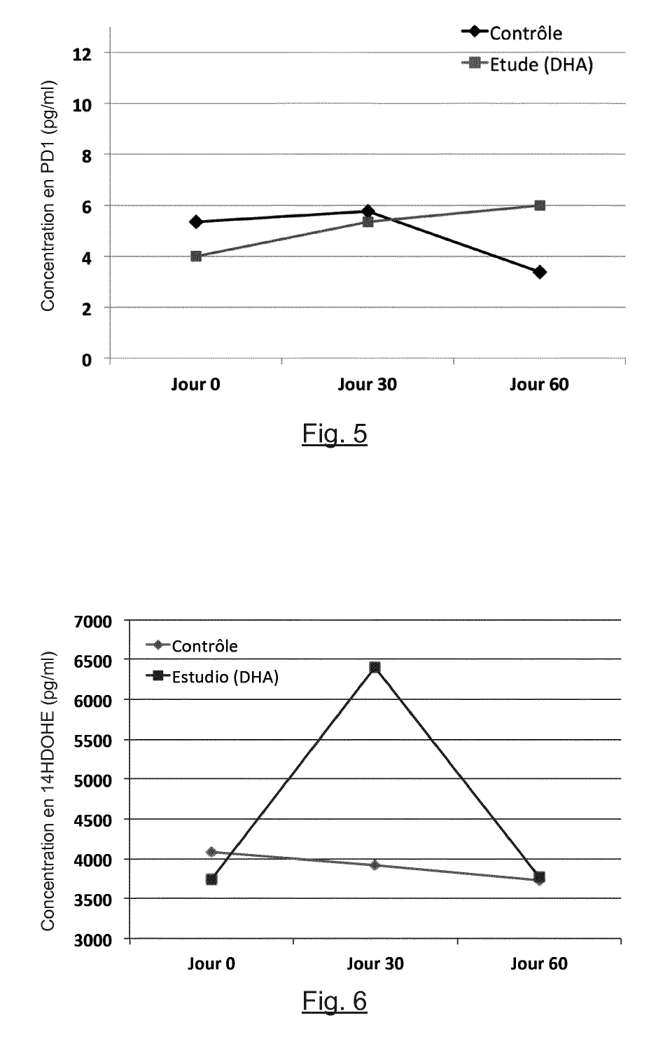

It can be seen in FIG. 4 that the study group had a leptin level twice as much as that of the control group. Leptin plays a role, among others, in the regulation of appetite and homeostasis of the immune response. Leptin is over-expressed among overweight or obese patients. As can be seen, the leptin level in subjects from the control group was reduced to a third because of their weight loss and loss of body fat mass. The patients in the study group on the contrary obtained more satisfactory results because their leptin level was on average reduced to one-fifth through the daily absorption of DHA (p<0.001 at 60 days). FIGS. 5 and 6 present the results obtained in the improvement in the expression of the anti-inflammatory markers PD1 and 14-HDOHE. Both groups show an increase of the level of these markers in the blood. As can be seen in the graph of FIG. 5, the PD1 rate is not improved among patients in the control group despite their weight loss: it is even reduced by 36% from the start of their diet. This result does not imply that inflammation progresses in the patient but that it lasted and became chronic without moving towards a resolution. On the contrary, patients having received DHA during their diet have their PD1 level gradually increase (+83% of PD1on day 60) as compared with day 0). This result shows that the dietary compositions including DHA not only act as a brake on obesity-related inflammation but also make the inflammation evolve towards its resolution. It can also be seen from FIG. 6 that subjects consuming DHA regularly had a 14-HDOHE level significantly higher than subjects in the control group (p<0.00095) at the end of 30 days.

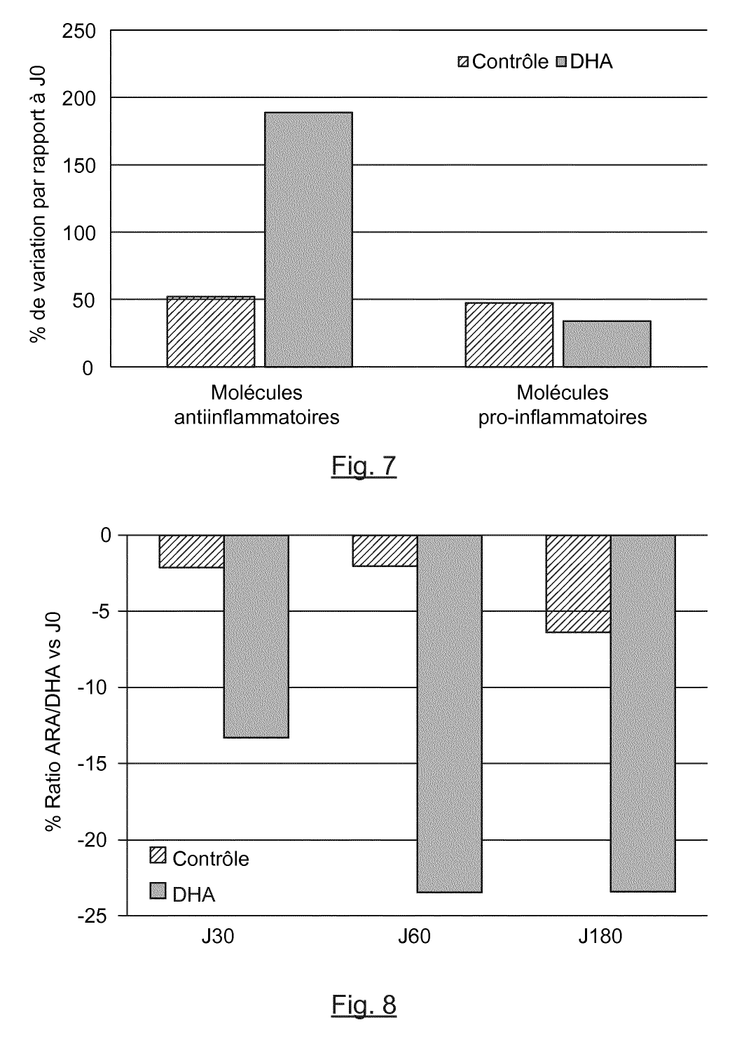

FIG. 7 presents the overall and average progress of the pro-inflammatory and anti-inflammatory markers for each group of patients after 6 months of slimming diet. To put it briefly, each patient had a blood test to evaluate the concentration of the different markers before starting his or her slimming diet. At the end of 6 months, the patients again underwent a blood test to measure the same markers. As can be seen from FIG. 7, patients who had received a daily supplement of DHA had a level of anti-inflammatory molecules about four times higher than that of the control group. Similarly, taking DHA also reduced the quantity of inflammation markers as compared with the control group.

FIG. 8 presents the progress of the ratio between arachidonic acid (denoted as ARA) and DHA over time, as compared with the initial ratio measured among patients before the start of their diet. Arachidonic acid (ARA) is a key metabolite of inflammation. This non-essential fatty acid serves as a pre-cursor for many inflammation markers. As can be seen in FIG. 8, as of the first thirty days of slimming, the ARA/DHA ratio became negative for both groups of patients. However, this diminishing is far greater and marked among patients consuming DAH. Indeed, from the results presented even after 180 days of dieting, it can be seen that the ARA/DHA ratio among patients in the control group has a level of reduction of about 50% as compared with that of the patients with DHA on D-30. The presence of DHA in the study group hampers lipolysis and therefore inhibits the increased secretion of fatty acids including arachidonic acid, thus reduces inflammation.

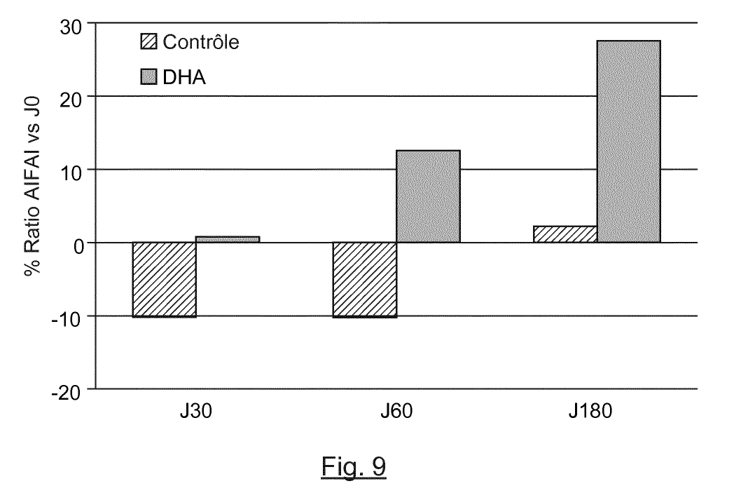

These figures corroborate those presented in FIG. 9. The anti-inflammatory fatty acid index (AIFAI) represents the total quantity of fatty acids having anti-inflammatory activity. According to the graph of FIG. 9, the AIFAI of patients in the control group was negative and stable during at least the first 60 days of the diet. It became slightly positive at the end of 6 months of dieting. Conversely, the AIFAI of patients having received a DHA supplement showed zero and slightly positive AIFAI as of the first month of dieting. It then increased regularly and became more than twelve times greater than that of the patients in the control group.

### CONCLUSION

These results clearly show that the intake of food compositions and food portions according to the invention, containing DHA, further reduces inflammation among obese subjects. More precisely, weight loss alone does not resolve the problem of low-grade inflammation observed among obese or overweight patients. As can be seen from the results presented in FIGS. 1 and 2, DHA does not modify the total weight loss. Nor does it accelerate weight loss. However, the daily consumption of DHA considerably reduces low-grade inflammation among patients. Hence, those subjects who followed a slimming diet based on the compositions according to the invention will t be less likely to regain weight at the end of their diet than patients in the control group. In addition, the risks of developing certain pathologies such as type 2 diabetes and cardiovascular problems are further reduced.

1. Ready-to-use diet food composition to reduce low-grade inflammation in an obese or overweight patient comprising, as a proportion of the total weight of the composition:
50% to 80% by weight of protein;
2% to 20% by weight of carbohydrates; and,
2% to 15% by weight of lipids;
said lipids including 0.25% to 2% by weight of docosahexaenoic acid (DHA) relative to said weight of lipids.

2. Ready-to-use diet food composition according to claim 1 comprising, relative to the total weight of the composition:
70% to 80% by weight of protein;
4% to 6 0% by weight of carbohydrates; and,
2% to 15% by weight of lipids.

3. Individual diet food portion to reduce low-grade inflammation in an obese or overweight patient comprising:
12.5 g to 20 g, preferably 14 g to16 g, of protein and,
0.5 g to 5 g, preferably 1 g to 5 g of carbohydrates,
0.25 to 6 g, preferably 0.5 g to 4 g of lipids; said lipids including up to 80 mg of DHA.

4. Individual diet food portion according to claim 3 comprising 1 mg to 80 mg of DHA, preferably 10 mg to 50 mg of DHA, in a more preferred way 25 mg to 50 mg of DHA and in a manner preferred among all, 50 mg of DHA.

5. Individual diet food portion according to claim 3, said portion having a weight of 5 mg to 300 mg, preferably 15 g to 200 g, and in a more preferred manner 20 g to 125 g.

6. Individual diet food portion according to claim 3, characterized in that it is made out of culinary ingredients, at least one of which contains DHA.

7. Individual diet food portion according to claim 6 wherein said culinary ingredient containing DHA is chosen from amongst egg yolk, fish oil, seaweed extract, mushrooms, krill or a combination of at least two of these ingredients.

8. Individual diet food portion according to claim 3, wherein said protein is protein chosen from among milk proteins, whey or milk serum, soya, egg or a combination thereof.

9. Diet food composition according to claim 1, obtained by mixing an Individual diet food portion with water.

10. Diet food composition according to claim 1, obtained by associating an individual died food portion with at least one DHA capsule, said capsule containing 100 mg to 300 mg of DHA.

11. Kit for a very-low-calorie type of diet comprising at least one dietary food portion according to claim 3, comprising at least one DHA capsule.

12. Use of DHA to prepare a dietary food composition to reduce low-grade inflammation in an obese or overweight patient, comprising, relative to the total weight of the composition:
50% to 80% by weight of protein;
2% to 20% by weight of carbohydrates; and
2% to 15% by weight of lipids;
said lipids including 0.5% to 15% of docosahexaenoic acid (DHA) by weight relative to said weight of lipids.

13. Use of DHA according to claim 12 to prepare a dietary food composition to reduce low-grade inflammation in an obese or overweight patient, comprising, relative to the total weight of the composition:
70% to 80% by weight of protein;
4% to 6% by weight of carbohydrates; and
2% to 5% by weight of lipids;
said lipids including 10% to 15% by weight of docosahexaenoic acid (DHA).

14. Individual diet food portion according to claim 3, characterized in that it is used by the patient for a period of 30 days to 180 days by the patient, preferably a period of 60 days to 180 days, and more preferably a period of 90 days to 180 days.

15. Food composition according to claim 1, characterized in that it is used by the patient for a period of 30 days to 180 days, preferably a period of 60 days to 180 days, and more preferably a period of 90 days to 180 days.
